(19) United States
(12) Patent Application Publication
(10) Pub. No.: US 2022/0160627 A1
(43) Pub. Date: May 26, 2022

(54) ENHANCED LICORICE ROOT CHEWS

(71) Applicant: MOUNTAIN LAKEHOUSE HOLDINGS, LLC, Houston, TX (US)

(72) Inventors: ADAM RODNEY, NEW YORK, NY (US); ADRIAN LARREA, WILMINGTON, NC (US)

(73) Assignee: MOUNTAIN LAKEHOUSE HOLDINGS, LLC, Houston, TX (US)

(21) Appl. No.: 17/533,362

(22) Filed: Nov. 23, 2021

Related U.S. Application Data
(60) Provisional application No. 63/117,958, filed on Nov. 24, 2020.

(51) Int. Cl. A61K 9/00; A24F 47/00; A23L 33/105; A61K 31/522; A61K 31/465; A61K 31/05

(52) U.S. Cl. CPC A61K 9/0056; A24F 47/00; A61K 31/05; A61K 31/522; A61K 31/465; A23L 33/105

(57) ABSTRACT

A long acting licorice root segment includes a licorice root segment infused or otherwise combined with an active agent and providing administration of the active agent to the oral cavity while the licorice root segment is in a mouth of a user. The active agent may include caffeine, a cannabinoid, or nicotine, or combinations thereof, and may also include additives such as flavorings, vitamins, mineral supplements, herbal supplements, and combinations thereof. Methods for preparing the infused licorice root segments are also described.

This application claims priority to U.S. Provisional Application Ser. No. 63/117,958, entitled ENHANCED LICORICE ROOT CHEWS, filed Nov. 24, 2020, the entire disclosure of which is hereby incorporated by reference.

### BACKGROUND OF THE INVENTION

The roots and the bark of the roots Licorice root, or Glycyrrhiza, are naturally sweet and contains about 4 percent glycyrrhizin acid which is 50 times sweeter than cane sugar (sucrose). Licorice root builds energy and is an excellent digestive tonic. It is a broad-spectrum natural detoxifying agent, ridding the body of over 1200 known toxins without any distressful side-effects. It also helps regulate the blood sugar level, helping counteract hypoglycemia, a very common imbalance. Licorice is a perennial herb native to southern Europe, Asia, and the Mediterranean.

Chewing on licorice roots sticks is popular in certain parts of the Caribbean. Patients chew licorice and enjoy its sweet taste. It is not addictive and is a safe substitute for a cigarette habits.

### SUMMARY OF THE PRESENT INVENTION

In accordance with various embodiment of the present invention, Licorice root, or Glycyrrhiza, is formulated as a licorice root segment in stick, cylindrical or tubular form as a safe and pleasant, noncombustible delivery system for a variety of substances. In this regard, these licorice root may be infused or otherwise combined with these substances pursuant to the process described below. In this regard, the term “licorice root” refers herein to licorice root in its fibrous root form, as contrasted with licorice root extract. A licorice root segment is a segment of such licorice root.

In accordance with a first embodiment of the present invention, a licorice root segment is infused with caffeine in order to provide a long-lasting stimulant chew. For example, a caffeine infused licorice root segment may be infused with 100-300 mg caffeine, preferably about 150 mg, and provides administration of the caffeine for an hour or more as the user sucks and chews on the caffeine infused licorice root segment. In addition to caffeine, the root segment can be infused with other additives such as flavorings, herbal extracts, and the like.

In accordance with a second embodiment of the present invention, a licorice root segment is infused with nicotine in order to provide a long-lasting stimulant chew. For example, a nicotine infused licorice root segment may be infused with 3-10 mg nicotine, preferably about 5-7 mg, and provides administration of the nicotine for an hour or more as the user sucks and chews on the nicotine infused licorice root segment. In addition to nicotine, the root segment can be infused with other additives such as flavorings, herbal extracts, and the like.

In accordance with a third embodiment of the present invention, a licorice root segment is infused with a cannabinoid in order to provide a long-lasting delivery of the cannabinoid. For example, a cannabinoid infused licorice root segment may be infused with 30-100 mg, preferably about 50 mg, of CBD, THC or other cannabinoid, and provides administration of the CBD or THC for an hour or more as the user sucks and chews on the cannabinoid infused licorice root segment. In addition to cannabinoids, the root segment can be infused with other additives such as flavorings, herbal extracts, and the like, and/or with caffeine or nicotine to provide a stimulant.

In accordance with a fourth embodiment of the present invention, a process for preparing a caffeine infused licorice root segment is provided, comprising obtaining a licorice root, cutting the licorice root into licorice root segments having a generally cylindrical shape, a length of 3-7 inches and a diameter of 0.2 to 0.4 inches. The licorice root segments are then submerged in a solution, dispersion or suspension of caffeine and a liquid to infuse caffeine into the licorice root segment in a desired amount. The liquid may for example, be water, grain alcohol, ethanol, glycerol, and combinations thereof. The desired amount may be 100-300 mg caffeine.

In accordance with a fifth embodiment of the present invention, a process for preparing a nicotine infused licorice root segment is provided, comprising obtaining a licorice root, cutting the licorice root into licorice root segments having a generally cylindrical shape, a length of 3-7 inches and a diameter of 0.2 to 0.4 inches. The licorice root segments are then submerged in a solution, dispersion or suspension of nicotine and a liquid to infuse nicotine into the licorice root segment in a desired amount. The liquid may for example, be water, grain alcohol, ethanol, glycerol, and combinations thereof. The desired amount may be 5-10 mg nicotine.

In accordance with a sixth embodiment of the present invention, a process for preparing a cannabinoid infused licorice root segment is provided, comprising obtaining a licorice root, cutting the licorice root into licorice root segments having a generally cylindrical shape, a length of 3-7 inches and a diameter of 0.2 to 0.4 inches. The licorice root segments are then submerged in a solution, dispersion or suspension of a cannabinoid and a liquid to infuse the cannabinoid into the licorice root segment in a desired amount. The liquid may for example, be water, grain alcohol, ethanol, glycerol, and combinations thereof. The desired amount may be 30-100 mg cannabinoid.

In accordance further aspects of the above-referenced embodiments, as the user chews on the root, the substance infused into the root (e.g. CBD, THC, caffeine, nicotine) is partially released and partially driven into the remaining portion of the root, thus providing a greater dose as chewing continues. In accordance with further aspects of these embodiments, a portion of the dose can be formulated as a faster releasing component, for example, by drilling one or more holes in the root, and filling them with the substance in the form of a resin, gel, or compacted powder. In addition or alternatively, one or more of the holes may include a flavoring substance. In other and further embodiments, a hole can be drilled longitudinally through some or all of the root, so provide for increased absorption.

### BRIEF DESCRIPTION OF THE DRAWINGS

The features and advantages of the present invention are illustrated by the attached drawings and are explained in the description of the present invention and in the claims.

FIG. 1 illustrates licorice root chewing sticks.

FIG. 2 illustrates an embodiment of the enhanced licorice chew stick that has been drilled along the axis for increased absorption of the infused compounds.

FIG. 3 illustrates an end view of the enhanced licorice chew stick that has been drilled along the axis, where he bark, body of the licorice root, and the area drilled along the axis (3) are shown.

FIG. 4 illustrates an embodiment of the present invention with multiple enhanced licorice chew sticks

FIG. 5 illustrates a consumer using an enhanced licorice chew stick

FIG. 6 illustrates an infused licorice root chew where a hole has been drilled perpendicular to one end of the enhanced licorice chew stick to create a cavity for placement of a flavor capsule or other materials into the enhanced licorice chew stick.

### DETAILED DESCRIPTION

The enhanced or infused licorice root chews in accordance with various embodiments of the present invention are an innovative use of licorice root in its natural or refined state to provide a delivery system for caffeine, nicotine, CBD, or THC. Nothing is burned, heated, or vaporized to use infused licorice root chews. The dangerous and potentially carcinogenic tars and other combustion products of cigars, cigarettes and e-cigarettes are not created by infused licorice root chews according to the present invention.

As used herein, “licorice root” or “licorice root in its natural or refined state” refers to the root form of licorice root, as contrasted with licorice root extract, which is a powder. The licorice root maybe cut, or debarked, washed, etc., and is still licorice root in its natural state as that term is used herein. A licorice root segment is a segment of a licorice root, which may or may not have had its bark removed.

The enhanced or infused licorice root chews are made of Licorice Root—Glycyrrhiza Glabra, a sweet herb that possesses significant anti-inflammatory properties. It is contemplated that at least four varieties of licorice root may be used for infused licorice root chews, Glycyrrhiza Glabara, Glycyrrhiza Uralensis, Glycyrrhiza Inflata and Glycyrrhiza Echinata.

The enhanced or infused licorice root chews may be made by a novel process of preparing a solution, suspension or dispersion of the desired infusion ingredient—caffeine, nicotine, CBD, or THC—in a liquid medium such as water, ethanol, grain alcohol, glycerin, glycerol, or combinations thereof. Preferably, a solution is prepared, and the liquid medium is a solvent. Most preferably, the solvent for caffeine is water, and the solvent for nicotine, CBD, and THC is ethanol/glycerol or grain alcohol/glycerol.

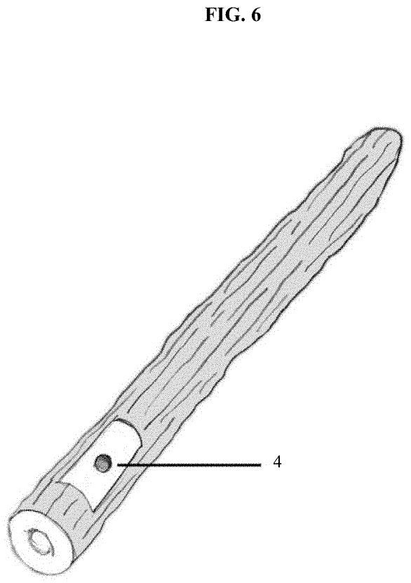

The caffeine, nicotine, CBD, THC, or other infusion ingredient may itself in liquid form, for example, CBD oil, THC oil, liquid caffeine, or liquid nicotine. Alternatively, the caffeine, nicotine, CBD, THC, or other infusion ingredient may be in solid form when it is combined with the liquid to form the solution, suspension or dispersion. The infused licorice root chews provide a long-lasting delivery of the caffeine, nicotine, CBD, THC, or other infusion ingredient into the oral cavity as the infused licorice root chew is placed in the mouth of the user (see FIG. 5) and chewed. Preferably, the infusion ingredient is delivered for an hour more. The amount of infusion ingredient contained in each infused root segment is preferably as follows: 100-300 mg, preferably about 150 mg for caffeine, 3-10 mg, preferably 5-7 mg for nicotine, and 30-100 mg, preferably about 50 mg, for CBD or THC.

To infuse the licorice roots, the licorice roots (or licorice root segments) are placed in the solution, suspension or dispersion for a determined period of time and then dried at a specified temperature. As a final step, a sample of each batch is tested using analytical chemistry methods and equipment to assure that the specified caffeine, nicotine, CBD, or THC concentration is present. The amount of caffeine, nicotine, CBD, or THC in the infused licorice root chews can be precisely controlled by varying the concentration of the caffeine, nicotine, CBD or THC in the solution or suspension or dispersion, the time that the licorice root soaks in the solution, suspension or dispersion, and the temperature. During the drying step, the water, grain alcohol, ethanol, glycerin, or glycerol is removed from the infused licorice root chews by evaporation. This controlled process permits the production of infused licorice root chews the ability to adjust the concentration of caffeine, nicotine, CBD, or THC in response to legal or governmental regulation, scientific or medical standards.

Infused licorice root chews may also be enhanced with various additional substances to further enhance the anxiety calming properties of CBD or CBD oils and the energy boosting properties of caffeine, caffeine oils or nicotine or nicotine oils. For example, the infusion could include, in addition to CBD, nicotine, THC and or caffeine, substances such as St. Johns Wort, Ginseng, Valerian, Chamomile, and other herbal remedies. CBD and THC infused licorice root chews can also be used for smoking cessation. In alternative and further embodiments, infused licorice root chews may be flavored with organic flavorings, spices, natural flavorings, or synthetic flavorings, including but not limited to cherry, grape, mango, orange, vanilla, chocolate, ginger, cinnamon, mint, lemon, lime, berry, and peppermint.

Infused licorice root chews are infused with the Caffeine, CBD, THC, Nicotine or other ingredients through the steps of: 1) dissolving the CBD, THC, Nicotine, caffeine or other licorice roots in the solution, for a specific time, at a desired temperature; 3) removing the licorice roots from the solution; and 4) removing the solvent by evaporation.

Infused licorice root chews are selected, sorted, cut, processed, manufactured, and packaged to provide a consistent, high quality consumer experience of chewing a botanical product for pleasure and health benefits.

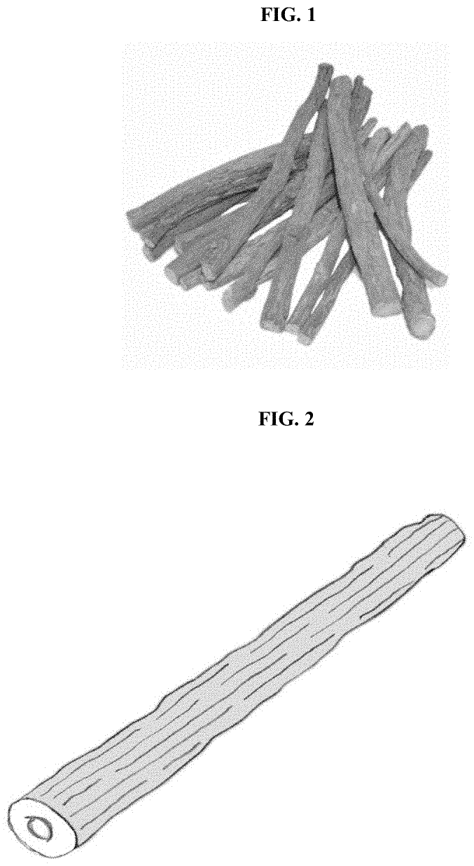

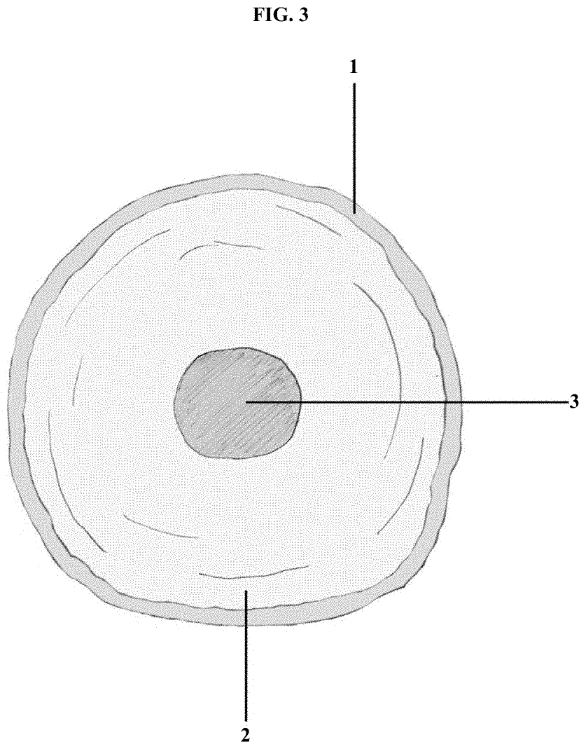

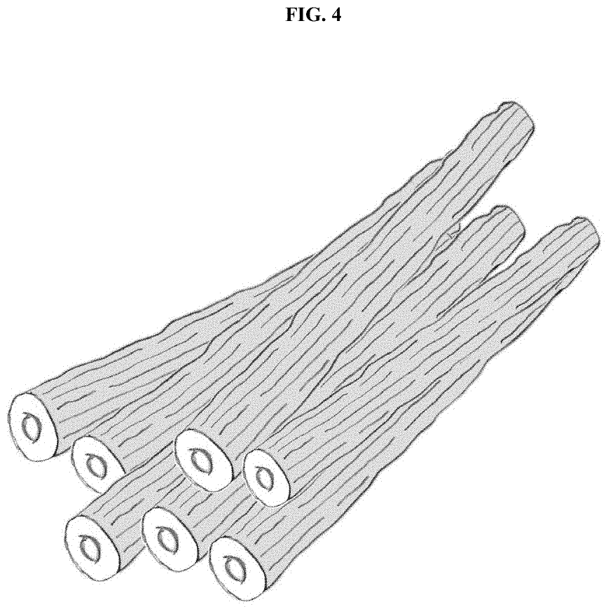

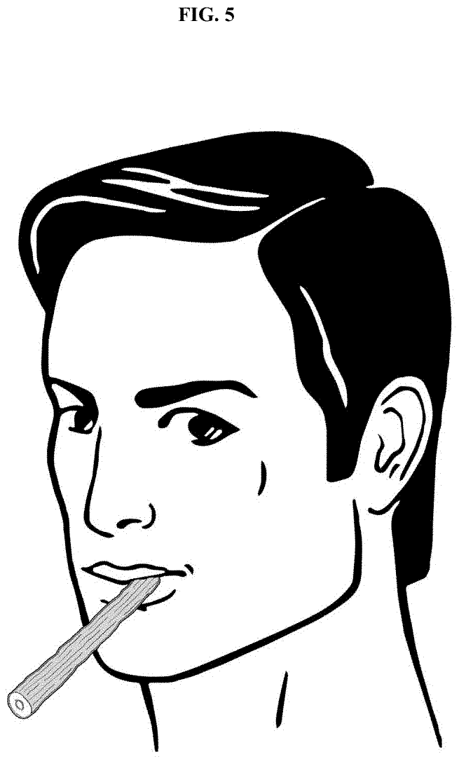

In one embodiment, the enhanced or infused licorice root chews, incorporates the bark of the licorice root or other botanical. In other embodiments of the enhanced or infused licorice root chews, the bark of the licorice root or other botanical has been peeled, milled or otherwise removed from the licorice root or other botanical. In any event the licorice root is cut into segments having a generally cylindrical shape, a length of 3-7 inches, preferably 3-5 inches, and a diameter of 0.2 to 0.4 As a person of ordinary skill in the art will appreciate, the segments are “generally cylindrical” in that as a naturally occurring root, they will have an irregular, though generally cylindrical shape. If desired, a hole can be drilled longitudinally through some or all of the root, to provide for increased absorption. FIG. 1 illustrates a set of licorice root segments. FIGS. 2 and 3 illustrate a licorice root segment having bark 1, a body 2, and a longitudinal hole 3 along the axis of the root. FIG. 4 shows a set of seven of the licorice root segments of FIGS. 2 and 3.

One embodiment of the enhanced or infused licorice root chews is infused or otherwise combined with a cannabinoid (hereinafter “CBD”), containing one or more of cannabidiol, cannabinol, dodeca-2E,4E,8Z,10E/Z-tetraenoic-acid-isobutylamides, cannabigerol, cannabichromene, cannabicyclol, cannabivarin, tetrahydrocannabivarin, cannabidivarin, cannabichromevarin, cannabigerovarin, cannabigerol monomethyl ether, cannabidiolic acid (CBDA), or a combination thereof. CBD due has recently become desired by consumers, many of whom do not want to smoke CBD. The presently-available CBD beverages, oils, creams and food additives do not provide a chewing experience. This invention addresses that need by enabling the consumer to enjoy botanical roots and enjoy CBD.

In another embodiment the enhanced or infused licorice root chews and cannabinoid additives are provided as a kit comprising separate portions of the infused licorice root chews and cannabinoid extracts, enabling users to combine the infused licorice root chews and cannabinoid additives in proportions of their choosing.

In another embodiment, the enhanced or infused licorice root chews are infused or otherwise combined with delta-9 tetrahydrocannabinol (“THC”) for medicinal use or recreational use as an alternative to liquid, edible or smokable THC products.

In another embodiment, the enhanced or infused licorice root chews are infused or otherwise combined with one or more vitamins to provide an alternative method of consuming vitamins.

In another embodiment, the enhanced or infused licorice root chews are infused or otherwise combined with one or more mineral supplements to provide an alternative method of consuming mineral supplements.

In another embodiment, the enhanced or infused licorice root chews is infused with one or more herbal supplements to provide an alternative method of consuming herbal supplements.

In another embodiment of the enhanced or infused licorice root chews, a hole is drilled along the longitudinal axis of the licorice root for increased absorption of the infused compounds. In this regard, the hole provides increased surface area and an interior passage forming an additional delivery path.

Referring to FIG. 6, in another embodiment of the enhanced or infused licorice root chews a hole 4 is drilled perpendicular or in a transverse (as opposed to longitudinal) direction to one end of the enhanced licorice chew stick to create a cavity for placement of a flavor capsule or other materials into the enhanced licorice chew stick. Preferably, the hole is perpendicular to the longitudinal axis and extends only part-way through the diameter of the root segment as illustrated in FIG. 6. However, the transverse hole could be, for example, 35 to 145 degrees from longitudinal, preferably 70-110 degrees, more preferably 80-100 degrees. In this regard, the transverse hole can incorporate a portion of the dose of the active agent (e.g. caffeine, nicotine, cannabinoid) to provide a faster delivery of that portion of the dose. Additives such as flavorings, taste masking substances, or other substances may also be included. For example, the caffeine, nicotine, cannabinoid, or other ingredients can be prepared as a resin, and inserted into the transverse hole. Other delivery vehicles known in the art to provide delivery in the presence of saliva could also be used, for example, a compacted powder, a tablet, an extruded pellet or pellets, etc., which are shaped to fit in the hole. As the user chews on one end of the licorice root chew, some of the active agent is delivered to the oral cavity, and some of the active agent is driven towards the opposite end of the root chew. As a result, the amount delivered increases as the user successively chews on portions of the chew having successively higher concentration of the active agent. Providing one or more transverse holes holding an additional dose of the active agent can serve to provide an additional portion of the dose early in the chewing process so as to provide a more consistent dose throughout the chewing process.

As noted above, the perpendicular or transverse hole (or holes) may include a flavor capsule or flavor pod. A licorice chew stick can also include a plurality of perpendicular or transverse holes, with for example, one hole holding the flavor capsule, and other holes holding active agent. The flavor capsule or flavor pod can be formulated in any manner known in the art to provide delivery in the presence of saliva. For example, For example, the flavoring combined with a binders (e.g., gums, starches, HPMC, PVP, or ethyl cellulose) and compressed or tableted into an appropriate shape to fit securely into the hole. Alternatively, the flavoring can be incorporated into a hard-candy vehicle, for example mixing the flavoring with sugar and corn syrup and then solidifying in a die, where the resulting flavor pod is shaped to fit securely into the hole. Alternatively, the flavoring pod can be formulated as part of a resin including the flavoring, or as extruded pellets including the flavoring.

The infused licorice root chews could, in other embodiments, be infused with other substances to alter or enhance the texture, taste or other properties of the present invention.

The Caffeine, Nicotine, CBD, THC and other substances are infused into the infused licorice root chews using, alternatively, immersion in a liquid slurry, liquid, chemical, electrical, electrolysis, absorption or other infusion processes. Although infusion is preferred, the Caffeine, Nicotine, CBD, THC and other substances could alternatively be combined or with the licorice root through other impregnation techniques known in the art.

The enhanced or infused licorice root chews, in other embodiments, are selected and cut to a specific length and packaged, individually or in packs with multiple products to make it easier for the consumer to carry and use the product.

The enhanced or infused licorice root chews, in other embodiments are provided in a package with a resealable closure.

### EXAMPLES

The three compounds (caffeine, nicotine, CBD) were diluted from analytical standards and infused into licorice root sticks using the process as described below. The final determination of amount infused is performed by UHPLC-MS/MS after extraction. The Limit of Quantitation (LOQ) for the three compounds is 0.20 mg/g. As used herein, LOQ means Limit of Quantitation; QC means Quality Control, SD means Standard Deviation; UHPLC means Ultra High Performance Liquid Chromatography and LC-MS/MS means Liquid Chromatography Tandem Mass spectrometry.

Test and Reference Substance Information

The infusion ingredients and test equipment used were as follows:

TABLE 1

| Caffeine | Nicotine | CBD |
|---|---|---|
| IUPAC name: 1,3,7- | IUPAC name: 3-[(2S)-1- | IUPAC name: 2-[(1R,6R)- |
| trimethylpurine-2,6-dione | methylpyrrolidin-2-yl]pyridine | 3-methy1-6-prop-1-en-2- |
| Empirical formula: | Empirical formula: | ylcyclohex-2-en-1-y1]-5- |
| C₈H₁₀N₄O₂ | C₁₀H₁₄N₂ | pentylbenzene-1,3-diel |
| Molar mass: 194.19 g/mol | Molar mass: 162.23 g/mol | Empirical formula: |
| Exact mass: 194.08 g/mol | Exact mass: 162.12 g/mol | C₂₁H₃₀O₂ |
| CAS number: 58-08-2 | CAS number: 54-11-5 | Molar mass: 314.5 g/mol |
| | | Exact mass: 314.22 g/mol |
| | | CAS number: 13956-29-1 |

TABLE 2

| | | Brand/ |
|---|---|---|
| Equipment | Size, Description | Manufacturer |
| Waterbath | 1225 digital water bath | VWR |
| Variable or Volumetric pipettes | various μL and mL | Fisher |
| | sizes | |
| Centrifuge Tube | 15 mL and 50 mL sizes | Karter |
| Autosampler vial with screw caps | 2 mL | Cogita |
| Balance, Top loading | PM300 or PJ300 | Mettler |
| Balance, Analytical | AE260 | Mettler |

### Reagents, Chemicals, Solvents, and Solutions

The reagents, chemicals solvents and solution used were as follows:

TABLE 3

| | | Brand/ |
|---|---|---|
| | | Manufacturer |
| Chemical/Solvent/Solution | Grade or components | or composition |
| Glycerol | ACS | Sigma-aldrich |
| Grain Alcohol | Food Grade | Everclear |
| Acetonitrile | UV/HPLC | Pharmco-Aaper |
| water | HPLC grade | Pharmco-Aaper |
| formic acid | Certified ACS or better | various |
| Ammonium formate | Certified ACS or better, | Sigma-aldrich |
| | glacial | |
| UHPLC Mobile Phase A | water/ACN/ammonium | 98/2/10 v/v/mM |
| | formate | |
| UHPLC Mobile Phase B | acetonitrile/water/formic | 98/2/0.1 v/v/v |

### Preparation of Standard Solutions

The standard solutions were prepared according to the following procedures:
1. Caffeine standard Solutions (various concentrations)
   Prepare a caffeine Stock Solution from solid analyte by diluting with HPLC water to 15 mg/mL into an appropriate vessel and vortex to mix. Any concentration above 15 mg/mL will not fully dissolve in solution at room temperature. A concentration of 50 mg/mL is fully soluble at 60 C and above. Concentration can vary depending on infusion target.
2. Nicotine Stock Solution (200 mg/mL)
   Prepare a nicotine Stock Solution by diluting 99% (−)-nicotine in glycerin to 200 mg/mL in appropriate amber glass vessel, paying careful attention to safety protocols. Vortex to mix.
3. Nicotine Standard Solution (various concentrations)
   Prepare further Standard Solutions of nicotine by diluting the Nicotine stock solution into food grade ethanol to required concentrations. Vortex to mix.
   Concentration can vary depending on infusion target.
4. CBD standard Solution (various concentrations)
   Prepare a 30 mg/mL CBD Standard Solution from solid analyte by diluting with 1:1 Glycerol:Everclear (v/v) into an appropriate vessel, vortex to mix, and heat to 60° C. in a hot water bath. Concentration can vary depending on infusion target.
5. Storage of Reference, Stock and Standard Solutions
   Reference, Stock and Standard solutions of compounds used for infusion or standard curve QC standards should be stored at 4° C. Caffeine will reenter solution after chilled if heated back to 60° C.

### Test Procedure Summary

The following is a summary of the test procedure which was followed:
1. Caffeine Infusion method for Licorice Root Stick at 150 mg per stick: Weigh sticks before beginning infusion. Submerge sticks into heated solution of 50 mg/ml caffeine solution inside appropriate vessel in hot water bath (60° C.). Leave sticks in water, weighing periodically. Remove sticks from solution after the stick had gained approximately 3.2 grams. Dry outside and weigh again to determine precise amount of solution absorbed within stick using concentration equation. Slight expansion and stick growth may be observed.
2. Nicotine Infusion method for Licorice Root Stick at 4 mg per stick: Weigh sticks before beginning infusion. Submerge sticks into appropriate vessel containing heated solution of 4 mg/ml nicotine in 1:1 gycerol:Everclear (v/v) in hot water bath (60° C.). Leave sticks in water, weighing periodically. Remove sticks from solution after the stick had gained approximately 1.0 grams. Dry outside and weigh again to determine precise amount of solution absorbed within stick using concentration equation. Slight expansion and stick growth may be observed.
3. CBD Infusion method for Licorice Root Stick at 40 mg per stick: Weigh sticks before beginning infusion. Submerge sticks into appropriate vessel containing heated solution of 30 mg/ml CBD in 1:1 gycerol:Everclear (v/v) in hot water bath (60° C.). Leave sticks in water, weighing periodically. Remove sticks from solution after the stick had gained approximately 1.25 grams. Dry outside and weigh again to determine precise amount of solution absorbed within stick using concentration equation. Slight expansion and stick growth may be observed.
4. Storage of sticks after infusion: Dry stick fully in oven at low temperature (for example 80-100° F.) to remove excess liquid and store in vacuum sealed container.

With regard to the above procedures it has been found that Glycerol addition to solution leads to better stick coloration and texture after infusion procedure. Glycerol addition may affect solubility of Caffeine. Shaking, mixing, or stirring during infusion may speed up absorption while also more quickly removing the sticks tannins. Higher temperature incubations (e.g. 200° F., for example) may be used to speed up absorption, but in cases where ethanol solutions are used, the higher absorption speed should be balanced against safety concerns as the temperature rises

Theoretical concentrations of analyte per stick were calculated as follows:

$$Concentration of analyte in individual stick = \frac{( Gend - Gint )}{Density} * Conc$$

where Gend is the final weight of stick after drying [g], Gint is the initial weight of stick [g]. Density is density of the infusion solution; and Conc is the concentration of analyte in infusion solution. Actual concentrations obtained from UHPLC-MS/MS after extraction. The results were as follows:

TABLE 4
Comparison of concentrations

| | | | | |
|---|---|---|---|---|
| | Theoretical | Actual | Ratio | |
| ID | conc. | conc. | diff | Medium |

Caffeine

| | | | | |
|---|---|---|---|---|
| 200012-017 | 5.01 | 8.67 | 1.73 | 15 mg/ml caffeine in H20 |
| 200012-018 | 10.91 | 7.78 | 0.71 | 15 mg/ml caffeine in H20 |
| 200012-019 | 9.87 | 8.14 | 0.82 | 15 mg/ml caffeine in H20 |
| 200012-024 | 140.42 | 128.66 | 0.92 | 50 mg/ml caffeine in H20 |
| 200012-025 | 141.38 | 156.95 | 1.11 | 50 mg/ml caffeine in H20 |
| 200012-026 | 125.11 | 113.29 | 0.91 | 50 mg/ml caffeine in H20 |

CBD

| | | | | |
|---|---|---|---|---|
| 200012-030 | 4.31 | 6.53 | 1.52 | 20 mg/ml Ethanol:glycerol |
| 200012-031 | 8.38 | 9.23 | 1.10 | 20 mg/ml Ethanol:glycerol |
| 200012-033 | 17.52 | 11.18 | 0.64 | 20 mg/ml Everclear |
| 200012-034 | 22.97 | 28.43 | 1.24 | 20 mg/ml Everclear:glycerol |
| 200012-035 | 19.90 | 16.49 | 0.83 | 20 mg/ml Everclear:glycerol |
| 200012-036 | 23.39 | 9.88 | 0.42 | 20 mg/ml Ethanol:glycerol |
| 200012-042 | 48.29 | 72.71 | 1.51 | 30 mg/ml Everclear:glycerol |
| 200012-043 | 80.61 | 75.15 | 0.93 | 30 mg/ml Everclear:glycerol |
| 200012-044 | 57.79 | 64.53 | 1.12 | 30 mg/ml Ethanol:glycerol |

Nicotine

| | | | | |
|---|---|---|---|---|
| 200012-037 | 4.64 | 8.04 | 1.73 | 4 mg/ml Everclear:glycerol |
| | | | | Base |
| 200012-038 | 2.75 | 3.46 | 1.26 | 4 mg/ml Everclear:glycerol |
| | | | | Base |
| 200012-039 | 4.18 | 3.82 | 0.91 | 4 mg/ml Everclear:glycerol |
| 200012-040 | 5.40 | 5.05 | 0.94 | 4 mg/ml Everclear:glycerol |
| 200012-041 | 4.16 | 4.83 | 1.16 | 4 mg/ml Everclear:glycerol |

The above results show that caffeine, CBD and nicotine can be successfully infused into licorice root segments.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A long acting enhanced licorice root segment combined with an active agent which includes caffeine, nicotine, and/or a cannabinoid, the licorice root segment having a length of 3 to 7 inches, a diameter of 0.2 to 0.4 inches, and providing administration of the active agent to the oral cavity while the licorice root segment is in a mouth of a user.

2. The long acting infused licorice root segment of claim 1, wherein the licorice root segment is infused with 100-300 mg caffeine, the licorice root segment providing administration of the caffeine to the oral cavity for an hour or more while the licorice root segment is in a mouth of a user.

3. The long acting infused licorice root segment of claim 1, wherein the licorice root segment is infused with 3-10 mg nicotine, the licorice root segment providing administration of the nicotine to the oral cavity for an hour or more while the licorice root segment is in a mouth of a user.

4. The long acting infused licorice root segment of claim 1, wherein the licorice root segment is infused with 30-100 mg of a cannabinoid, the licorice root segment providing administration of the cannabinoid to the oral cavity for an hour or more while the licorice root segment is in a mouth of a user.

5. The long acting infused licorice root segment of claim 4, wherein the cannabinoid is selected from the group consisting of cannabidiol, cannabinol, dodeca-2E,4E,8Z,10E/Z-tetraenoic-acid-isobutylamides, cannabigerol, cannabichromene, cannabicyclol, cannabivarin, tetrahydrocannabivarin, cannabidivarin, cannabichromevarin, cannabigerovarin, cannabigerol monomethyl ether, tetrahydrocannabinol (THC), tetrahydrocannabinolic acid (THCA), or tetrahydrocannabivarin (THCV).

6. A process for preparing a plurality of infused licorice root segment according to claim 2, comprising
providing one or more licorice roots,
cutting the one or more licorice roots into licorice root segments having a generally cylindrical shape, a length of 3-7 inches and a diameter of 0.20 to 0.40 inches;
submerging the licorice root segments in a solution, dispersion or suspension of caffeine and a liquid to infuse a desired amount of caffeine into each licorice root segment, the desired amount being an amount between 100-300 mg of caffeine.

7. The process according to claim 6, wherein the liquid is water, grain alcohol, ethanol, glycerol, or combinations thereof.

8. The process according to claim 6, further comprising drying the licorice root segments.

9. A process for preparing a plurality of infused licorice root segment according to claim 3, comprising
providing a licorice root, cutting the licorice root into licorice root segments having a generally cylindrical shape, a length of 3-7 inches and a diameter of 0.2 to 0.40 inches;
submerging the licorice root segments in a solution, dispersion or suspension of nicotine and a liquid to infuse a desired amount of nicotine into each licorice root segment, the desired amount being an amount between 3-10 mg of nicotine.

10. The process according to claim 9, wherein the liquid is water, grain alcohol, ethanol, glycerol, or combinations thereof.

11. The process according to claim 9, further comprising drying the licorice root segments.

12. A process for preparing a plurality of infused licorice root segment according to claim 4, comprising
providing a licorice root, cutting the licorice root into licorice root segments having a generally cylindrical shape, a length of 3-7 inches and a diameter of 0.2 to 0.40 inches;
submerging the licorice root segments in a solution, dispersion or suspension of the cannabinoid and a liquid to infuse a desired amount of the cannabinoid into each licorice root segment, the desired amount being between 30-100 mg of the cannabinoid.

13. The process according to claim 12, wherein the liquid is water, grain alcohol, ethanol, glycerol, or combinations thereof.

14. The process according to claim 12, further comprising drying the licorice root segments

15. The enhanced licorice root segment of claim 1, wherein the licorice root segment is cut from a licorice root selected from Glycyrrhiza Glabra, Glycyrrhiza Uralensis, Glycrrhiza Inflataand and Glycrrhiza Echinata.

16. The enhanced licorice root segment of claim 15, wherein the licorice root segment does not include bark.

17. The enhanced licorice root segment of claim 1, further comprising an additive selected from the group consisting of flavorings, vitamins, mineral supplements, herbal supplements, and combinations thereof.

18. The enhanced licorice root segment of claim 1, further comprising a hole is along the longitudinal axis of the licorice root segment for increased absorption of the active agent.

19. The enhanced licorice root segment of claim 1, further comprising a hole is drilled perpendicular to one end of the enhanced licorice chew stick, and having a portion of the active agent located therein.

20. The enhanced licorice root segment of claim 1, further comprising a hole is drilled perpendicular to one end of the enhanced licorice chew stick, and having a flavoring substance located therein.
